(19) United States
(12) Patent Application Publication
(10) Pub. No.: US 2006/0094995 A1
(43) Pub. Date: May 4, 2006

(54) Bandage material with active carbon fibers

(76) Inventor: Vaclav Bauer, Dobris (CZ)

Correspondence Address: ARNOLD & FERRERA, L.L.P., 2401 FOUNTAIN VIEW DRIVE, SUITE 630, HOUSTON, TX, 77057, US

(21) Appl. No.: 11/034,187

(22) Filed: Jan. 12, 2005

(30) Foreign Application Priority Data
Nov. 1, 2004 (CZ) PV 2004-1086

(51) Int. Cl. A61F 5/00

(52) U.S. Cl. 602/1

(57) ABSTRACT

A bandage material with active carbon fibers is disclosed, in which the bandage material is comprised of a load-bearing layer made of an unwoven viscose/polypropylene textile material, and an active layer made of 200 g/m² pure carbon fiber. When combined, the load-bearing layer and the active layer form a single compact union. A bandage material suitable for use on a bleeding wound or other, smaller surface wounds, etc., is comprised of a load-bearing layer, which can have any flat shape and in a preferred embodiment houses bandage material with active carbon fibers, oriented with an active carbon fiber layer disposed on top, and with the edges of the load-bearing layer fitted with an adhesive layer to form a peel-away protective cover.

### FIELD OF THE INVENTION

The present invention relates generally to health-care bandaging materials, and in a specific, non-limiting embodiment, to a bandage material having active carbon fibers suitable for the dressing of wounds.

### BACKGROUND OF THE INVENTION

Many types of bandage materials are used to dress wounds in the health-care sector. For example, dry materials are used to cover wounds, and other materials impregnated with various salves or other means are used to increase the closing speed of the wound. However, such materials are inappropriate for purulent wounds, because they cling to the wound, and when replacement is necessary, old bandages must be torn away; thereby slowing down the healing process. For purulent and other wounds that heal slowly, a bandage material containing active carbon is more appropriate for its adsorbent qualities.

VLIWAKTIV is one example of the multilayer bandage materials present in the market. It consists of an outer layer, which protects the clothes, a cellulose layer, a cloth made of viscose with active carbon, and a special layer applied directly to the wound. It is known that active carbon absorbs smells, neutralizes unpleasing odors of infected and septic wounds. Active carbon also can neutralize bacteria. However, the adsorption layer of the VLIWAKTIV bandage material, i.e., compressed carbon particles, is not in direct contact with the wound area, and thus is not active. The layer in direct contact with the wound clings to it, and thus slows down the healing process.

Another commercially available material, ACTISORB® PLUS, has a layered structure similar to VLIWAKTIV, including active carbon enriched with silver. This bandage material adsorbs the secretion into a cellulose layer, and the silver contained in the active carbon layer destroys germs. In this case, the carbon layer is also on a substrate. The carbon layer is not in direct contact with the wound, which once again reduces the effectiveness of this bandage material, and again the bandage clings to the wound.

Each of the above-mentioned bandage materials has their active carbon layer or carbon fibers encapsulated, which reduces their effectiveness. Moreover, each of the above-mentioned bandage materials is thick and difficult to manipulate. Finally, where known carbon fibers, whether woven or unwoven, have been used as an active layer, they have always been encapsulated. Thus, no known bandage material has applied an active carbon or carbon fiber layer directly to the surface of a wound.

### SUMMARY OF THE INVENTION

A bandage material with active carbon fibers is disclosed in which the bandage material comprises a load-bearing layer made from a non-woven textile material containing viscose and polypropylene; and an active layer containing pure carbon fiber, wherein said load-bearing layer and said active layer are formed into a compact unit. In some embodiments, the load-bearing layer and the active layer are bonded together using a heat-activated process. In other embodiments, the heat-activated process further comprises activation by means of a thermally reactive powder.

In other embodiments, the active layer covers at least a portion of the load-bearing layer. In still other embodiments, the bandage material further comprises a plurality of layers having a combination of load-bearing layers and active layers. In still further embodiments, the bandage material is oriented with an active carbon fiber layer disposed on top, and edges of the load-bearing layer are fitted with an adhesive protective layer.

In further embodiments, the bandage material comprises a facemask having fittings to allow attachment to the ears or the head, wherein said facemask comprises at least one layer of bandage material having active carbon fibers. In further embodiments, the facemask comprises an active carbon fiber layer that is covered by a protective layer on either side, thereby forming a single compact unit. In still further embodiments, the facemask has one or more layers folded over longitudinally. In yet further embodiments, the facemask is reinforced on its sides, and opens in a middle portion to allow insertion of a mouth and nose.

### BRIEF DESCRIPTION OF THE DRAWINGS

FIG. 1 depicts a bandage material according to the present invention.

FIG. 2 depicts the bandage material with active carbon fibers arranged in a rectangular shape and with an adhesive perimeter.

FIGS. 3a-3c depicts the bandage material with active carbon fibers arranged as a facemask, where FIG. 3a is the front view, FIG. 3b is a cross section showing how the facemask is formed, and FIG. 3c is a cross section without folds.

### DETAILED DESCRIPTION

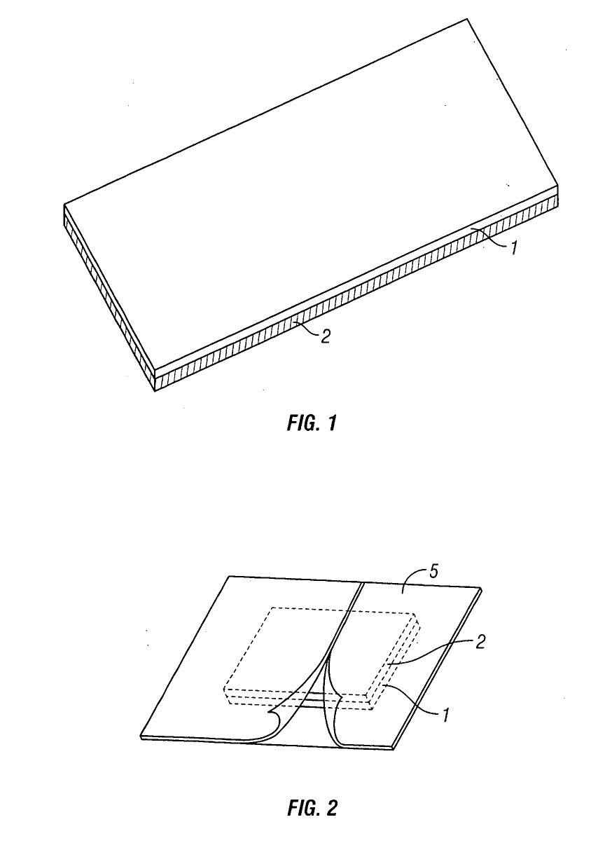

The above-mentioned disadvantages in the prior art can be greatly reduced using the proposed bandage material with active carbon fibers, which, in the presently preferred embodiment depicted in FIG. 1, comprises a load-bearing layer 1 made of an unwoven viscose/polypropylene textile material, and an active layer 2 made of woven or unwoven carbon fiber layers, wherein the load-bearing layer and the active layer are joined to form a single compact union. In some embodiments, the load-bearing and active layers are bonded by a heat-activated process, e.g. welding, fusion, etc., using a non-toxic thermally reactive powder.

In one embodiment, load-bearing layer 1 further comprises a viscose and polypropylene combination. In some embodiments, the ratio of viscose to polypropylene is about 1:1, though other effective ratios will occur to those of ordinary skill in the art. In other embodiments, load-bearing layer 1 comprises polypropylene, viscose cuts or fibers, or a portion of viscose injected between the polypropylene and viscose layers at a higher temperature, thereby bonding the layers together. In various embodiments, the active layer 2 covers the entire load-bearing layer 1, or, in alternative embodiments, only covers parts of the load-bearing layer 1. The bandage material disclosed herein can also consist of several layers, for example, various combinations of load-bearing and active layers.

According to one embodiment, a layer of material with carbon fibers is disposed on top of a substrate. In other embodiments, the layer of material with carbon fibers extends over the edges of the active layer, and an adhesive layer is applied to those non-active edges.

In the embodiment depicted in FIG. 2, the adhesive layer has a protective coating 5, which is peeled away before use, so that the active layer 2 can be attached directly to the wounded area, while the adhesive perimeter attaches to surrounding healthy skin. This type of bandage material with active carbon fibers is appropriate for use when dressing either bleeding wounds or smaller, deeper wounds. The disclosed bandage material with active carbon fibers is breathable, and if used with a non-breathable substrate, can be formed into any shape. For example, the bandage can easily be formed into a rectangle, with an adhesive layer exposed on all sides.

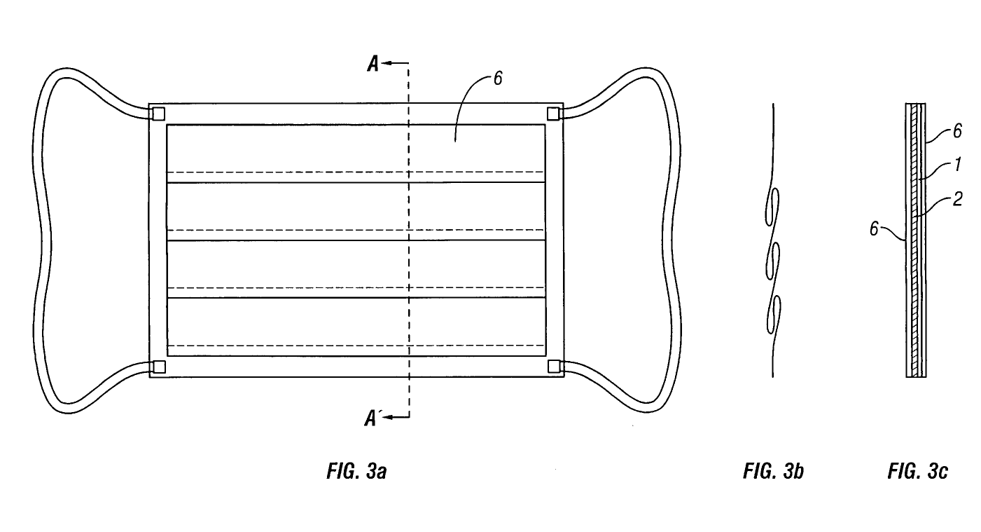

As seen in FIGS. 3a-3c, the carbon fiber material is formed into the shape of a disinfecting facemask suitable for protecting the mouth and nose. In the depicted embodiment, the bandage material is shaped to cover the mouth and nose, and includes at least one layer of carbon fiber bandage material according to the invention, and of protective layers 6 disposed on either side of the facemask. The side with active carbon fibers is applied to the mouth and nose. The facemask also has normal fittings that permit attachment on the ears or the head. Of course, the facemask can also be manufactured having only the active carbon fiber layer and the protective layers, but without the various fittings mentioned above.

Bandage material according to the invention has led to the creation of a safe load-bearing layer, which hosts an active layer made of carbon fibers, where the resulting compact unit allows the active layer to be laid directly onto the wound, as it does not separate nor break. This allows the positive properties of carbon fibers—high sorption speed and capacity for germs, toxins, chemical and toxic substances and odors—to be used. Since the bandage material facilitates re-granulation, the wound, when dressed with such materials, clears up and heals very quickly. The active layer made of carbon fibers can also be produced in different thicknesses. For example, the active layer can be very thin, and the bandage maintains its positive qualities even when formed very thin.

The weave made of carbon fibers is non-toxic, does not provoke adverse reactions, and is both breathable and hydrophilic, but with low water adsorption characteristics. Carbon weave contains no foreign substances that could irritate or reduce adsorption. The design and mechanical properties of the claimed bandage material allows direct contact between the carbon fibers and the wound surface, where the carbon fibers lend their adsorption capacity.

In some embodiments, the active layer is a weave made of hydrated cellulose fiber, subjected to controlled carbonization and activation at temperatures of several hundreds ° C. This thermic treatment results in a pure activated carbon shaped as an original weave, having a microporous structure of fibers, thereby resulting in a high capacity to adsorb organic matters. Carbon is extremely stable chemically and is relatively heat resistant. Carbon is also resistant to hydrolysis, photolysis, oxidation, and thermal decomposition at normal temperatures. Carbon requires high temperatures to combine with oxygen, sulphur, and other halides, and does not dissolve in most solvents; it will, however, dissolve in molten metals.

The bandage material of the invention is not primarily intended for absorption of secretions, as it has only a small absorption capacity. Instead, secretions are absorbed into another absorptive material applied over the bandage of the invention. The active layer does not cling to the wound. To the contrary, it can be left in place for long periods, because it heals and disinfects the wound and adsorbs odors. Accordingly, only the top absorptive material is normally replaced.

The bandage material according to the invention can be used in health care for a number of purposes, e.g., to treat trophic, purulent, complicated post-surgery wounds with bacterial contamination, etc.; it is also used to eliminate unpleasant odors. Advantages of the invention include the fact that the active layer can be applied directly onto the wound, and thus the effects are better than with currently known bandages, even those including carbon fibers. The instant bandage material is non-toxic, easy to apply, is not aggressive, and removes odors and actively traps germs and chemical agents. It helps to prevent secondary infections, speeds up the healing of treated surfaces, and generally will not lead to adverse effects when applied to a location. The bandage is also simple to apply, adapts well to the surface of the defect, and is sanitized and packaged according to healthcare standards.

Moreover, facemasks made of bandage material with active carbon fibers can be used, for the above-mentioned reasons, in chemically contaminated areas or operations instead of masks, provided that leaks around the nose and mouth are prevented. Facemasks according to the invention are therefore very suitable for wearing within areas harmful to health.

Returning to the specific, though non-limiting embodiment of the invention depicted in FIG. 1, an example bandage material comprises an inactive white load-bearing layer 1 made of non-woven textile, including one layer of about 38% viscose cuts, and another layer of about 50% polypropylene cuts and of about 12% liquid viscose, which is sprayed between the two layers during production, for a total weight of about 120 g/m²; Load-bearing layer 1 serves as a base for a second, active, black layer 2 made of, for example, about 200 g/m² pure carbon fiber, and is attached using non-toxic, thermally activated powder, so that the combined load-bearing and active layers form a single compact unit.

Active layer 2 of the bandage material is applied directly to the wound. Handling of this bandage is simple and the patient can handle it alone. The bandage material is highly hydrophilic, breathable, and permeable, and is very suitable for adsorption of germs and organic matter. It has only small absorption capacity, however, and is therefore not intended for absorption of secretions, which instead must be absorbed by another absorbent material, which usually is placed as a cover over the bandage material of the invention. Active layer 2 contains no organic fraction of other impurities subject to decay, and consists of virtually pure carbon with about <1% remaining after annealing.

The bandage material with active carbon fibers depicted in FIG. 2 comprises a substrate 3, which includes the bandage material with carbon fibers, and further consists of an inactive load-bearing layer 1, an active carbon fibers layer 2, and adhesive edges 4 of the substrate 3 disposed on the side with active carbon fibers. In some embodiments, adhesive edges are covered with a protective coating 5, which at the same time protects the active carbon fiber layer 2. When the protective coating 5 is peeled away, the bandage material with active carbon fibers is pressed onto the wound by its active side 2, and the adhesive edges are pressed onto surrounding healthy skin.

The bandage material with active carbon fibers depicted in FIGS. 3a-3c has a rectangular shape and consists of at least three layers, of which the middle is a bandage material with active carbon fibers consisting of inactive layer 1, an active carbon fiber layer 2, and is protected on both sides by a protective layer 6. The facemask has fittings that allow fastening to the ears or the head. The active carbon fiber surface of the facemask is applied to the mouth and nose, and is only lightly covered by one of the protective layers 6. The facemask can also be manufactured in a manner in which all layers are doubled over longitudinally and reinforced at the sides, with an opening provided in the middle to allow insertion of a user's mouth and nose.

The bandage material of the invention has broad application possibilities in the health care field. For example, it can be used in dermatology for shank sores, sores typical for diabetes mellitus, hard-to-heal wounds with secondary infection, open infected tumor defects, burns, post-surgery wounds, etc. Bandage materials having adhesive edges are more suitable for small bleeding wounds. Bandage materials with active carbon fibers shaped as facemask are suitable for any location where facemasks are worn, though a facemask manufactured in the manner described herein will be much more effective than other, previously known masks.

The foregoing specification is provided for illustrative purposes only, and is not intended to describe all possible aspects of the present invention. Moreover, while the invention has been shown and described in detail with respect to several exemplary embodiments, those of ordinary skill in the pertinent arts will appreciate that minor changes to the description, and various other modifications, omissions and additions may also be made without departing from either the spirit or scope thereof.

1. A bandage material with active carbon fibers, wherein said bandage material comprises:
a load-bearing layer made from a non-woven textile material containing viscose and polypropylene; and
an active layer containing pure carbon fiber;
wherein said load-bearing layer and said active layer are formed into a compact unit.

2. The bandage material of claim 1, wherein said load-bearing layer and said active layer are bonded together using a heat-activated process.

3. The bandage material of claim 2, wherein said heat activated process further comprises activation by means of a thermally reactive powder.

4. The bandage material of claim 1, wherein said active layer covers at least a portion of said load-bearing layer.

5. The bandage material of claim 1, further comprising a plurality of layers having a combination of load-bearing layers and active layers.

6. The bandage material of claim 1, wherein said load-bearing layer is oriented with active carbon fiber layer disposed on top, and wherein edges of said load-bearing layer are fitted with an adhesive protective layer.

7. The bandage material of claim 1, wherein said bandage material further comprises a facemask having fittings to allow attachment to the ears or the head, wherein said facemask comprises at least one layer of bandage material having active carbon fibers.

8. The bandage material of claim 1, wherein said bandage material further comprises a facemask having fittings to allow attachment to the ears or the head, wherein said facemask comprises an active carbon fiber layer that is covered by a protective layer on either side, thereby forming a single compact unit.

9. The bandage material of claim 1, wherein said bandage material further comprises a facemask having one or more layers folded longitudinally.

10. The bandage material of claim 9, wherein said facemask is reinforced on its sides, and opens in a middle portion to allow insertion of a mouth and nose.
